(19) United States
(12) Patent Application Publication
(10) Pub. No.: US 2019/0025492 A1
(43) Pub. Date: Jan. 24, 2019

(54) OPTICAL FIBER APPARATUS WITH HIGH DIVERGENCE ANGLE AND LIGHT SOURCE SYSTEM USING SAME

(71) Applicant: Corning Incorporated, Corning, NY (US)

(72) Inventor: Venkata Adiseshaiah Bhagavatula, Big Flats, NY (US)

(21) Appl. No.: 16/069,715

(22) PCT Filed: Jan. 27, 2017
(86) PCT No.: PCT/US2017/015292

§ 371 Date: Jul. 12, 2018

Related U.S. Application Data
(60) Provisional application No. 62/288,858, filed on Jan. 29, 2016.

(51) Int. Cl. F21V 8/00; G02B 6/02; A61B 3/00; A61B 1/07; A61B 1/313; A61F 9/007; C03B 37/15

(52) U.S. Cl. CPC G02B 6/0008; G02B 6/02295; A61B 3/0008; A61B 2090/306; A61B 1/313; A61F 9/00736; C03B 37/15; A61B 1/07

(57) ABSTRACT

A high-divergence-angle optical fiber apparatus is disclosed that includes a multimode optical fiber having a distal end and a divergence angle θ′. A light-redirecting structure is operably disposed at the distal end and consists of an array of between 1 and 10 layers of fused glass microspheres. The light-redirecting structure defines a divergence angle θ, wherein θ≥2θ′. A light source system that utilizes the high-divergence-angle optical fiber apparatus is also disclosed.

This application claims the benefit of priority under 35 U.S.C. § 119 of U.S. Provisional Application Ser. No. 62/288,858 filed on Jan. 29, 2016, the content of which is relied upon and incorporated herein by reference in its entirety.

### FIELD

The present disclosure relates to optical fibers and light sources, and in particular relates to an optical fiber apparatus that has a high divergence angle for use as a high-divergence-angle light source, and a light source system that uses the optical fiber apparatus.

### BACKGROUND

Optical fibers are used for a variety of applications to convey light from a light source to a remote location. While the main application of optical fibers is for optical telecommunications, optical fibers are also used as sources of illumination and are especially useful for illuminating regions that are difficult to access using conventional illumination systems and methods.

Optical-fiber illumination is defined in part by the divergence angle of the light leaving the optical fiber end. Generally, the divergence angle of an optical fiber is determined by the index difference between the core and cladding of the optical fiber and the index of refraction of the medium in which the light-emitting end of the optical fiber is immersed. In many applications, such as biomedical applications, the illumination needs to have a very high divergence angle, e.g., of about 90 to 100 degrees in air. This is particularly challenging in biomedical applications because the light-emitting end of the optical fiber is immersed in biological fluids, which have indices of refraction of about 1.33 (e.g., about that of water), while the silica fiber core has a refractive index of nominally 1.45.

For an optical fiber to have such a high divergence angle in media like water and biological materials, the index of refraction difference between the core and cladding needs to be very high, e.g., >0.25 or >0.3. To have such very high refractive index difference while meeting other illumination requirements is very difficult. For example, for high-brightness illumination applications, the optical fiber needs to withstand high power levels, e.g., several hundreds of milliwatts over the operating wavelength range, which for visible light is from 400 nm to 700 nm. In addition, wavelength-dependent absorption or scattering needs to be avoided, as does the damage threshold of the biological media in which the light travels.

In addition, in cases where the optical fiber is to be immersed in biological media, it is preferred that the optical fiber not have materials, such as adhesives or like binding materials, that can aversely react with the biological media or remain in the biological media after the optical fiber has been removed.

### SUMMARY

An aspect of the disclosure is a high-divergence-angle (HDA) optical fiber apparatus that consists of: a multimode optical fiber having glass core, a lower-index glass cladding surrounding the glass core, a proximal end and a distal end, wherein the optical fiber has a divergence angle θ′; a light-redirecting structure operably disposed at the distal end and consisting of an array of fused glass microspheres having diameters in the range from 3 microns to 25 microns, wherein the array is fused to the distal end and has between 1 layer and 10 layers of microspheres; and wherein the light-redirecting structure defines a divergence angle θ, wherein θ≥2θ′. In an example, a majority of the microspheres is within a size range from 3 microns to 5 microns.

Another aspect of the disclosure is the HDA optical fiber apparatus described above, wherein the optical fiber has a core diameter D1 of nominally 40 microns and a cladding outer diameter D2 of nominally 50 microns.

Another aspect of the disclosure is the HDA optical fiber apparatus described above, wherein the glass microspheres, the glass core and the glass cladding are each made of a silica-based glass.

Another aspect of the disclosure is the HDA optical fiber apparatus described above, wherein the array has between 1 layer and 6 layers of microspheres.

Another aspect of the disclosure is the HDA optical fiber apparatus described above, wherein the microspheres are solid.

Another aspect of the disclosure is the HDA optical fiber apparatus described above, wherein θ≥3θ′.

Another aspect of the disclosure is the HDA optical fiber apparatus described above, wherein the core has a diameter D1 and includes adjacent the distal end a widened core section that has a diameter D1′ that is at least 5% greater than the core diameter D1.

Another aspect of the disclosure is the HDA optical fiber apparatus described above, wherein the distal end of the optical fiber is curved.

Another aspect of the disclosure is the HDA optical fiber apparatus described above, wherein the distal end of the optical fiber includes depressions and protrusions that have a size substantially the same as the glass microspheres.

Another aspect of the disclosure is the HDA optical fiber apparatus described above, wherein the fused microspheres define air-filled interstices within the light-redirecting structure.

Another aspect of the disclosure is a light source system that includes the HDA optical fiber apparatus described above and a light source optically coupled to the proximal end of the optical fiber. The light source includes a light emitter that emits light that is coupled into the optical fiber, wherein the light is emitted from the light-redirecting structure as divergent light over the second divergence angle θ.

Another aspect of the disclosure is the light source system as described above, wherein the light emitter emits non-polarized visible light in the range from 440 nm to 650 nm.

Another aspect of the disclosure is a method of forming an HDA optical fiber apparatus using a multimode optical fiber having a glass core, a lower-index glass cladding surrounding the glass core, a proximal end and a distal end, wherein the optical fiber has a first divergence angle θ′. The method includes: arranging an array of glass microspheres adjacent the distal end, the microspheres having diameters in the range from 3 microns to 25 microns; and applying heat to the microspheres and to the distal end to fuse the microspheres to each other and to the distal end to form a light-redirecting structure that consists only of the microspheres and that defines a second divergence angle θ, wherein θ≥2θ′.

Another aspect of the disclosure is the method described above, and further including: prior to the act of applying heat, using a binding material to bind the microspheres to each other and to the distal end of the optical fiber; and wherein the act of applying heat includes burning off substantially all of the binding material.

Another aspect of the disclosure is the method described above, wherein the optical fiber has a core diameter of nominally 40 microns and a cladding outer diameter of nominally 50 microns.

Another aspect of the disclosure is the method described above, wherein the glass microspheres, the core and the cladding are made of a silica-based glass.

Another aspect of the disclosure is the method described above, wherein the array has between 1 layer and 6 layers of microspheres.

Another aspect of the disclosure is the method described above, wherein the microspheres are solid.

Another aspect of the disclosure the method described above, wherein the fused microspheres define air-filled interstices within the light-redirecting structure.

Another aspect of the disclosure is the method described above, wherein the core has a diameter D1 and further including a widened core section adjacent the distal end, the widened core section having a diameter D1′ that is at least 5% greater than the core diameter D1.

Another aspect of the disclosure is the method described above, and further including forming the distal end of the optical fiber to have a convex curvature.

Another aspect of the disclosure is the method described above, and further including forming protrusions and depressions on the distal end of the optical fiber, wherein the protrusion and depressions have a size substantially the same as the microspheres.

Another aspect of the disclosure is the method described above, and further including operably arranging a light source relative to the proximal end of the optical fiber and emitting a divergent light beam from the light-redirecting structure.

Another aspect of the disclosure is the method described above, wherein the light source emits non-polarized broadband visible light over a spectral range from 440 nm to 650 nm.

Another aspect of the disclosure is the method described above, and further including immersing the light-redirecting structure in a biological fluid.

Another aspect of the disclosure is a light source system that emits divergent light of a visible wavelength λ, the light source system including: a multimode optical fiber having a proximal end and a distal end, and a divergence angle θ′; a light-redirecting structure operably disposed at the distal end and consisting of an array of fused glass microspheres having diameters in the range from 3 microns to 25 microns, wherein the light-redirecting structure is fused to the distal end and has between 1 layer and 10 layers of microspheres and defines a divergence angle θ, wherein θ≥2θ′; and a light source optically coupled to the proximate end of the optical fiber and that emits light of the visible wavelength λ.

Additional features and advantages are set forth in the Detailed Description that follows, and in part will be readily apparent to those skilled in the art from the description or recognized by practicing the embodiments as described in the written description and claims hereof, as well as the appended drawings. It is to be understood that both the foregoing general description and the following Detailed Description are merely exemplary, and are intended to provide an overview or framework to understand the nature and character of the claims.

### BRIEF DESCRIPTION OF THE DRAWINGS

The accompanying drawings are included to provide a further understanding, and are incorporated in and constitute a part of this specification. The drawings illustrate one or more embodiment(s), and together with the Detailed Description serve to explain principles and operation of the various embodiments. As such, the disclosure will become more fully understood from the following Detailed Description, taken in conjunction with the accompanying Figures, in which:

FIG. 1 is as side view of a prior art optical fiber;

FIG. 2A is a side view of the high-divergence-angle (HDA) optical fiber apparatus as disclosed herein;

FIG. 2B is a close-up cross-sectional view of a bare end section of the HDA optical fiber apparatus of FIG. 2A, showing the light-redirecting structure at the fiber end, wherein the light-redirecting structure is formed by microspheres fused to the fiber end and to each other;

FIG. 3 is similar to FIG. 2B and is a closer view of the fiber end showing the microspheres being held together and to the fiber end by a binding material prior to being processed to form the light-redirecting structure of FIG. 2B;

FIGS. 4A and 4B are schematic side views of an example arc fusion system used to heat-process the fiber end, the microspheres and the binding material as shown in FIG. 3 to create the light-redirecting structure of FIG. 2B;

FIG. 5 is a schematic diagram of a laser system used to heat-process the fiber end, the microspheres and the binding material as shown in FIG. 3 to create the light-redirecting structure of FIG. 2B;

FIG. 6A is close-up cross-sectional view similar to FIG. 2B, and shows how guided light traveling in the optical fiber exits the fiber end and is redirected by the light-redirecting structure to increase the divergence angle of the emitted light;

FIG. 6B is a close-up cross-sectional view of an example microsphere illustrating how guided light from three different directions exits the fiber end and is focused by the microsphere along three different focus axes;

FIGS. 7A and 7B are similar to FIGS. 6A and 6B but for an example light-redirecting structure that uses hollow microspheres, with FIG. 7B showing the multiple refractions of light passing through the hollow microsphere to increase the divergence angle;

FIGS. 8A and 8B are similar to FIGS. 6A and 6B and illustrate an example wherein the fiber end includes depressions and protrusions that assist in increasing the divergence angle of light emitted from the fiber end;

FIG. 9A is a close-up, cross-sectional view of the bare end section of the HDA fiber apparatus, illustrating an example wherein the core includes a widened core section at the fiber end that increases the mode-field diameter of the guided light;

FIG. 9B is a close-up, cross-sectional view of the bare end section of the HDA fiber apparatus, illustrating an example wherein the fiber end has a curved surface;

FIG. 9C is a close-up, cross-sectional view of the bare end section of the HDA fiber apparatus, illustrating an example wherein the HDA fiber apparatus includes the widened core section of FIG. 9A and the curved surface of the fiber end of FIG. 9B;

FIG. 10 is a schematic diagram of an example light source system that includes the HDA fiber apparatus as disclosed herein; and

FIG. 11 is a schematic diagram of the light source system of FIG. 10 operably arranged relative to an eye for performing a biomedical procedure on the eye.

### DETAILED DESCRIPTION

Reference is now made in detail to various embodiments of the disclosure, examples of which are illustrated in the accompanying drawings. Whenever possible, the same or like reference numbers and symbols are used throughout the drawings to refer to the same or like parts. The drawings are not necessarily to scale, and one skilled in the art will recognize where the drawings have been simplified to illustrate the key aspects of the disclosure.

The claims as set forth below are incorporated into and constitute part of this Detailed Description.

Cartesian coordinates are shown in some of the Figures for the sake of reference and are not intended to be limiting as to direction or orientation.

In the discussion below, the phrase “between M and N” includes the values of M and N, e.g., “between 1 and 10 layers” includes 1 layer and also includes 10 layers.

The optical fiber “distal end” is also referred to herein as the “fiber end” or “fiber end face.”

In the discussion below, the divergence angle θ of the HDA optical fiber apparatus and the divergence angle θ′ of the optical fiber used in the HAD optical fiber apparatus are each measured from the central axis of the optical fiber. In an example, the divergence angle θ is measured as the full-width half-maximum (FWHM) of the intensity distribution of the emitted light, and in various examples the divergence angle θ as measured in air is greater than 40 degrees, or greater than 50 degrees, or greater than 60 degrees, or greater than 70 degrees, or greater than 80 degrees. Also in an example, the divergence angle θ is in the range from 40 degrees to 110 degrees, or 60 degrees to 90 degrees. The divergence angle θ as measured in a liquid is reduced as a function of the refractive index of the liquid. In an example, a “high divergence angle” θ is one that is at least twice the divergence angle θ′ of the optical fiber, i.e., θ≥2θ′.

In the discussion below, reference to an increase in the divergence angle θ is relative to the divergence angle θ′ of the optical fiber, i.e., the optical fiber alone, without the light-redirecting structure at the distal end of the optical fiber.

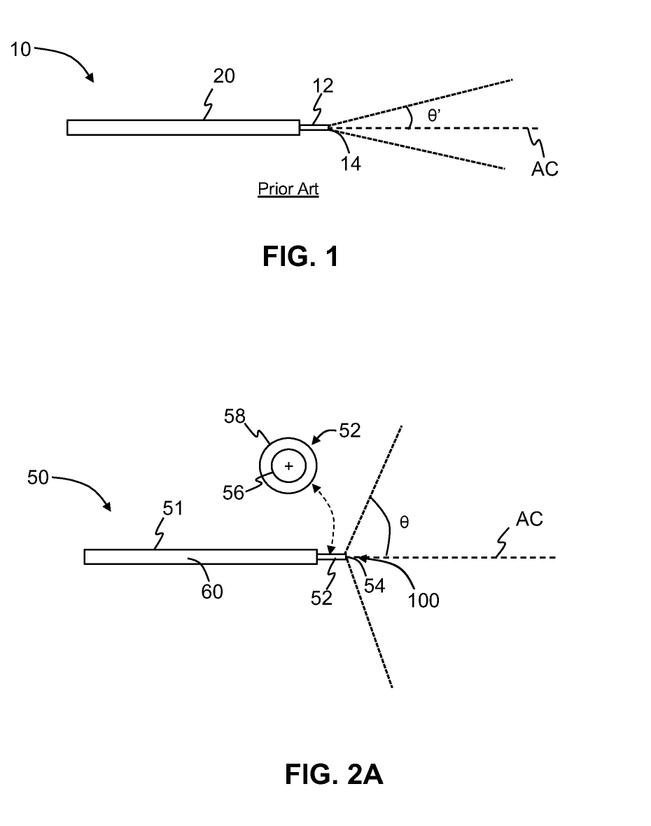

FIG. 1 is as side view of a prior art optical fiber 10 that includes a glass guiding section 12 having an end 14 and a central axis AC and a divergence angle θ′. The glass guiding section 12 is surrounded by a non-glass protective coating 20, such as a polymer. In the case where optical fiber 10 is multimode, the divergence angle θ′ is typically in the range from 12 degrees to 15 degrees when the optical fiber end 14 resided in air. When the optical fiber end resides in a liquid medium such as water, the divergence angle θ′ is reduced. Thus, the divergence angle θ′ of a conventional optical fiber is too narrow for many illumination applications, such as the aforementioned biomedical applications.

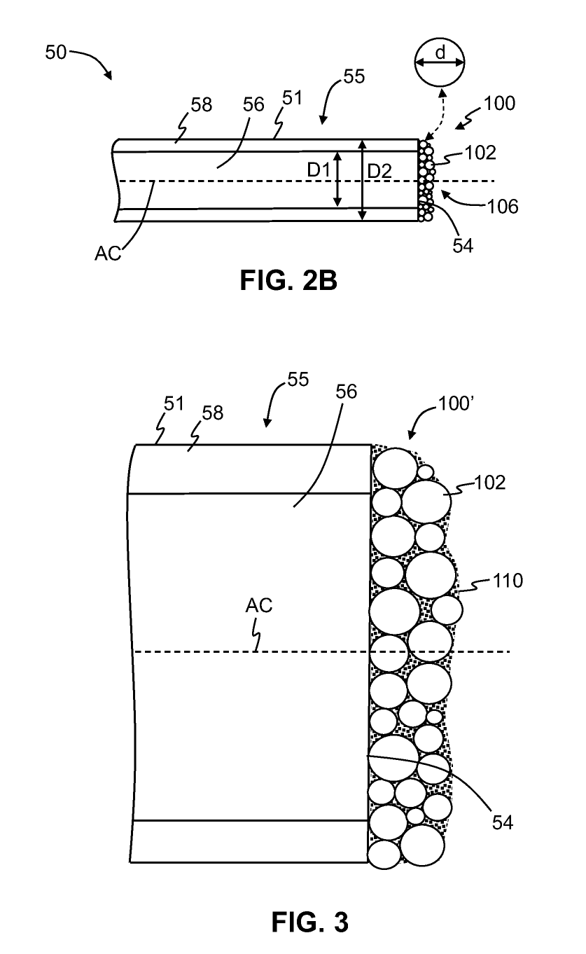

FIG. 2A is similar to FIG. 1 and shows an example high-divergence-angle optical fiber apparatus (“HDA fiber apparatus”) 50 as disclosed herein. The HDA fiber apparatus 50 includes an optical fiber 51 that has a glass guiding section 52 with a distal end (i.e., “fiber end” or “end face”) 54. The glass guiding section 52 is surrounded by a non-glass protective coating 60. The glass guiding section 52 includes a core 56 of refractive index n1 surrounded a cladding 58 of refractive index n2, wherein n1>n2 (i.e., the cladding is lower index), as shown in the close-up inset. In an example, core 56 is made of silica and can included doped silica, while cladding 58 can also be made of silica that has a lower refractive index than the core. In an example, core 56 is made of pure silica while cladding 58 is made of doped silica, e.g., fluorine-doped silica. In an example, optical fiber 51 is a prior-art optical fiber, such as optical fiber 10 of FIG. 1. The optical fiber 51 has a divergence angle θ′.

In an example, optical fiber 51 can be made of one or more of the following glasses: silica, soda-lime, titania, borosilicate glass (e.g., PYREX glass), alumina, and silicon oxynitride.

In an example, optical fiber 51 is a multimode fiber with a step refractive index and a core diameter D1 of nominally 40 microns and cladding outer diameter D2 of nominally 50 microns (see FIG. 2B). Here, the term “nominally” means to within the fabrication tolerance of the optical fiber. In an example, optical fiber 51 has a numerical aperture (NA=nₐ·Sin θ′) of about 0.26 in air (air index of refraction nₐ^{˜}1), which corresponds to a divergence angle θ′=sin⁻¹ (NA/nₐ)=0.263 rads or about 15 degrees. Other types of optical fiber 51 with different core diameters D1 and different refractive index profiles can also be effectively employed.

FIG. 2B is a close-up cross-sectional view of a bare end section 55 of HDA fiber apparatus 50 that includes glass guiding section 52 and fiber end 54 (i.e., there is no coating 60 in the bare end section). The HDA fiber apparatus 50 includes an array 100 of microspheres 102 that are fused to each other and to fiber end 54. In an example, microspheres 102 are made of glass, and further in the example are made of silica or a silica-based glass. In an example, microspheres 102 have a diameter d that is outside of the light-scattering regime for the wavelength λ of light used, so that the microspheres redirect the light mainly if not entirely by refraction. In an example, the wavelength λ of light is in the visible range and the diameter d of microspheres 102 is in the range from 3 microns to 25 microns.

The microspheres 102 need not all have the same size, i.e., a range of sizes can be used within the stated diameter range. Thus, in an example, microspheres 102 can have a size (diameter) distribution, with the majority of the microspheres having a diameter of between 3 and 10 microns or between 3 and 5 microns, or the average diameter of the microspheres is between 3 and 10 microns or between 3 and 5 microns. In an example, microspheres 102 are not perfect spheres, i.e., they are substantially spherical to within manufacturing tolerances. In an example, 10% or fewer of the microspheres in light-directing structure 106 can have a diameter smaller than 3 microns.

While glass microspheres 102 are generally preferred, the microspheres can be made of other materials, such as polymers (e.g., PMMA or polystyrene), ceramics or crystalline materials. The type of glass used can also vary, e.g., silica, soda-lime, borosilicate glass (e.g., PYREX), titania, alumina, silicon oxynitride, any one of the optical glasses known in the art, etc. The microspheres 102 can be solid or hollow, as described below. In an example, array 100 can consist of both solid microspheres and hollow microspheres.

The fusing of microspheres 102 to form array 100, as described in greater detail below, results in the formation of light-redirecting structure 106 that does not have any binding material to keep the microspheres attached to each other and to fiber end 54. Thus, in an example, there is no other material in bare end section 55 other than the glass of glass guiding section 52 and microspheres 102, and in particular there is no material used for binding, adhering, fusing, etc. the microspheres to each other and to fiber end 54 other than the glass of the glass guiding section 52 and the glass that makes up microspheres 102.

For strong and reliable fusing of microspheres 102 to fiber end 54, it is preferred that the coefficients of thermal expansion (CTEs) and the softening points of optical fiber 51 and microspheres 102 be similar. If the CTEs are significantly different, the microspheres 102 and/or the fiber end 54 can experience large stresses when heated and may cause fractures and degrade the adhesion of the microspheres to the fiber end. The stresses depend on the contact area, the CTE mismatch and the softening point. In an example, the softening points of fiber end 54 and microspheres 102 are within 300° C. to 500° C. of each other. In one example, the CTEs are within 5×10⁻⁶ of each other, while in another example the CTEs are within 1×10⁻⁶ of each other.

In an example, microspheres 102 are disposed on fiber end 54 in 1 to 10 layers, e.g., in 1 and 6 layers or in 2 to 3 layers. In an example, the layers can be relatively loose, i.e., not perfectly ordered or particularly well defined, especially in cases where the sizes of microspheres 102 vary.

Method of Making the Microsphere Array

FIG. 3 is another close-up view of bare end section 55 and shows an array 100′ of microspheres 102 as held together by a binding material 110, such as an organic adhesive (e.g., polymer-based adhesive). In an example, the microspheres 102 and binding material 110 are formed as a mixture and then applied to fiber end 54, e.g., by dipping the fiber end into the mixture or by spreading the mixture onto the fiber end. In an example, binding material 110 has a low viscosity that allows for a thin layer of the mixture and thus just a few layers of microspheres 102 to be applied to fiber end 54.

In an example, microspheres 102 can be substantially evenly distributed over fiber end 54 using a mixture in the form of a pH-controlled solution having dispersants. The binding material 110 can be an epoxy or a low-viscosity glue. In this case, the fiber end 54 can be dipped in a thin layer (e.g., <5 microns) of binding material 110 and then dipped in a substantially uniform layer of microspheres 102, which attach to the thin layer of binding material. The latter step can be performed by pressing fiber end 54 and the microspheres 102 adhered thereto onto a flat surface to spread the microspheres out substantially evenly, as well as to remove any excess layers of the microspheres.

Because optical fiber 51 and microspheres 102 are made of dielectric materials, in another example, electrostatic techniques can be used to attach the microspheres to fiber end 54 as an initial step prior fusing the microspheres to the fiber end and to each other. In an example, an electrostatic charge is created at fiber end 54 (e.g., by rubbing bare end section 55 with a cloth) and then bringing the charged fiber end into contact with microspheres 102.

Once microspheres 102 are disposed on fiber end 54 using the binding material 110 or electrostatic means, the array 100′ of microspheres (which can also include binding material 110), as well as the fiber end, are subjected to a heating process to fuse the microspheres to each other and to the fiber end. In the example where binding material 110 is used in array 100′, the heating process is used to burn off substantially all of the binding material (i.e., only trace amounts, if any, may remain). In this regard, glass-based optical fibers 51 are preferred over plastic or polymer optical fibers since the latter have lower softening temperatures and can be damaged during the heating process that removes the binding material.

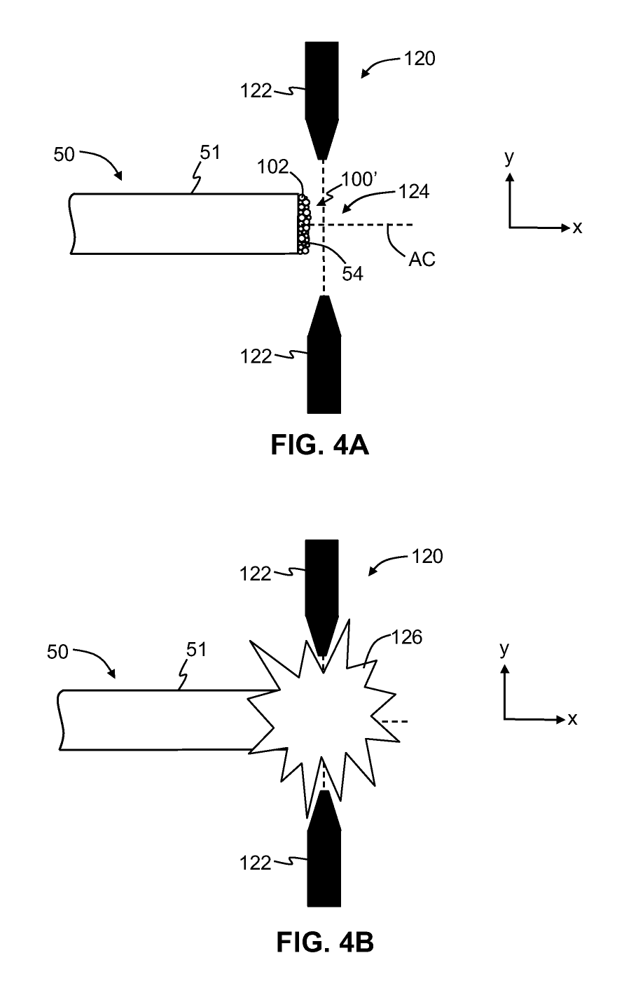

FIG. 4A is a side view of an example arc fusion system 120, which can be a standard fusion splicer. Cartesian x-y coordinates are shown for reference. The arc fusion system 120 includes spaced-apart opposing electrodes 122 arranged in the y-direction on respective sides of the x-axis, with a gap 124 between the electrodes. The fiber end 54, with array 100′ thereon, is inserted into or proximate to gap 124. Once fiber end 54 and array 100′ are in place, then with reference to FIG. 4B, arc fusion system 120 is activated so that the electrodes 122 generate a spark 126 in gap 124. The spark 126 represents a source of heat, wherein the amount of current provided to electrodes 122 defines the amount of heat generated by the spark.

In an example, a first spark 126 is used to burn off binding material 110 and to create an initial fusion of microspheres 102 to fiber end 54 and to each other. The heating process can then be repeated a number of times with additional sparks 126 to increase the fusing strength between microspheres 102 and to fiber end 54. In an experiment, an arc current of about 12 milliamps for an arc time of 250 milliseconds was used to fuse silica microspheres 102 (Bangs SS05N dry silica beads) having an average diameter d of 3.93 microns. The binding material 110 was a low-viscosity ultraviolet epoxy. A total of 4 sparks 126 were used in the heating process to perform the fusion and to burn off binding material 110. In an example, a single spark 126 can be used to process fiber end 54 and array 100′ by employing a higher arc current and a longer arc time. The result of the arc thermal processing is the formation of light-redirecting structure 106 of FIG. 2B.

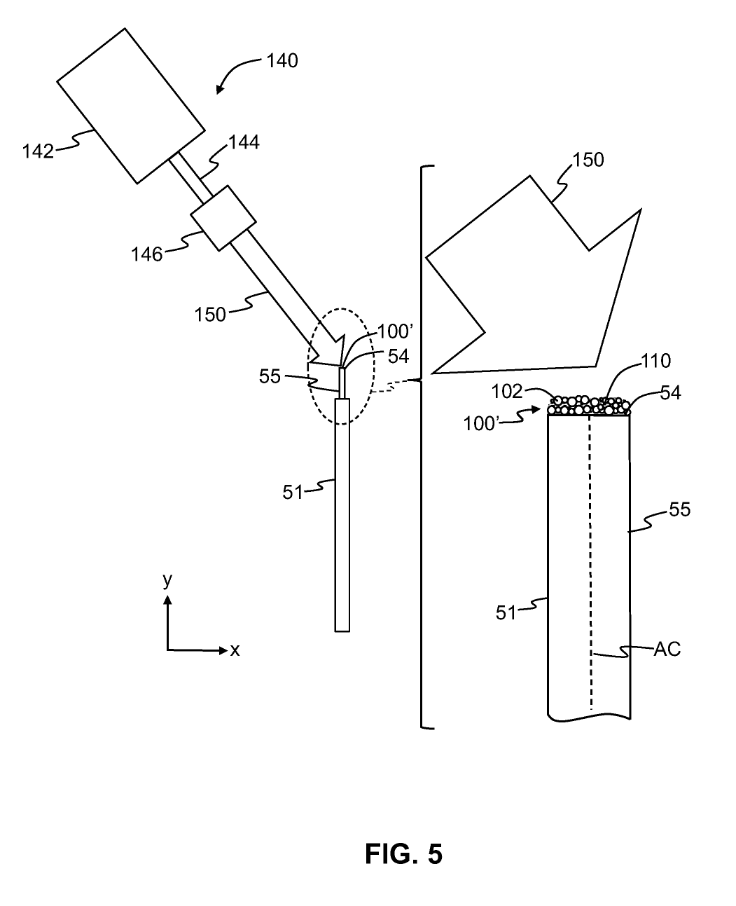

FIG. 5 is a side view of optical fiber 51 and array 100′ as shown in FIG. 3 operably arranged relative to a laser system 140. The laser system 140 includes a laser source 142 that emits an initial laser beam 144. The laser system 140 can also include a beam-conditioning optical system 146 that receives initial laser beam 144 and forms therefrom a processing laser beam 150. The optical fiber 51 is disposed so that processing laser beam 150 is incident upon fiber end 54 and array 100′. The processing laser beam 150 provides an amount of heat necessary to burn off binding material 110 while fusing the microspheres 102 to themselves and to fiber end 54.

In an example, laser source 142 is a CO₂ laser that emits at an infrared wavelength of nominally 10.6 microns or an ND-YAG laser that emits infrared wavelength of nominally 1.06 microns. Other lasers that emit a laser beam 144 that can heat and fuse microspheres 102 to each other and to fiber end 54 while burning off binding material 110 can be effectively employed.

In an example, laser source 142 emits light pulses so that the amount of energy in processing laser beam 150 can be precisely controlled, which in turn allows for the amount of heat delivered to array 100′ to be precisely controlled. The fusing process using process laser beam 150 can be carried out in a number of steps, e.g., a first step that burns off binding material 110 while performing an initial fusion of microspheres 102 to fiber end 54 and to each other, and then additional steps to increase the strength of the fusion of microspheres 102 to fiber end 54 and to each other. The fusion process can also be carried out in a single step that results formation of light-redirecting structure 106 of FIG. 2B.

Light Divergence by Refraction

As noted above, the size of microspheres 102 are selected so that they mainly if not entirely refract light rather than scatter light. This means that most if not all of microspheres 102 have a diameter d that is substantially larger than the wavelength of light used to form the divergent illumination. For example, if visible wavelengths of light are used (e.g., from 0.4 microns to 0.7 microns), then the diameter d of microspheres 102 is preferably at least four times the largest wavelength in the wavelength band, e.g., 2.8 microns. If a narrow-band green light having a wavelength of 0.5 microns is employed, then in an example, the diameter d of microspheres 102 is preferably at least 2 microns.

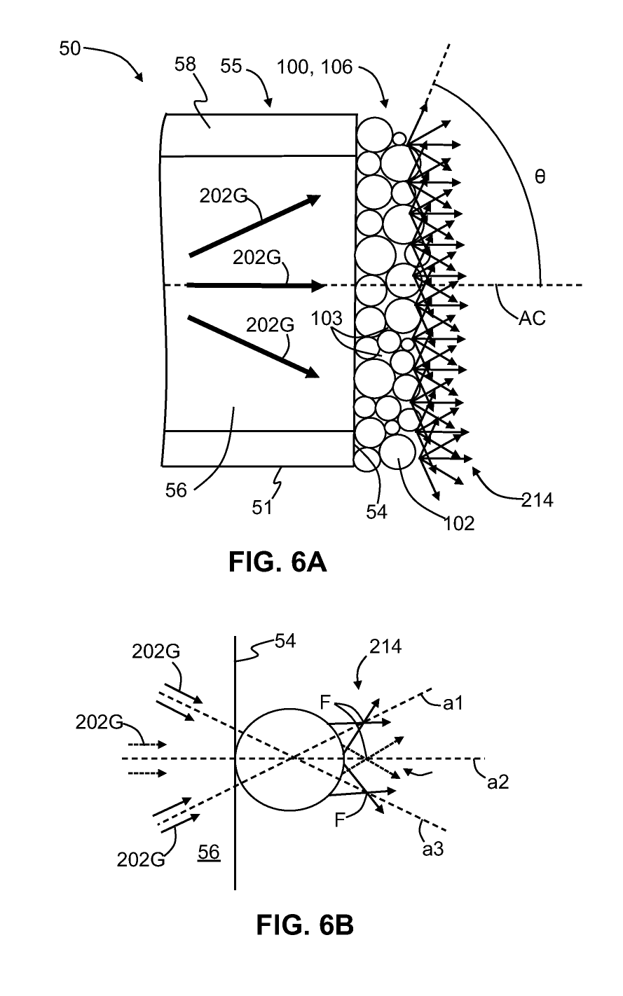

FIG. 6A is a close-up view of bare end section 55 of HDA fiber apparatus 50 that shows array 100 of fused microspheres 102 that define the light-redirecting structure 106 at fiber end 54. The fused microspheres 102 in array 100 define open (e.g., air-filled) interstices 103, which are discussed below. Also shown in FIG. 6A is guided light 202G traveling in core 56 of optical fiber 51 at different angles that represent different waveguide modes supported by optical fiber 51. In practice, a multimode fiber can include tens or hundreds of guided modes each traveling at a different angle, and only a few angles are shown in FIG. 6A by way of illustration. The guided light 202G exits fiber end 54 and is then refracted by each microsphere 102 in array 100, with the collective effect of the light-redirecting structure 106 being a divergent light beam 214 having divergence angle θ. In an example, the divergence angle θ is at least twice as large as the optical fiber divergence angle θ′, i.e., θ≥2θ′, while in another example θ≥2.5θ′, while in another example, θ≥3θ′.

FIG. 6B is a close-up view of a single solid microsphere 102 and schematically illustrates guided light 202G traveling in optical fiber 51 along different directions (angles), as indicated by respective focus axes a1, a2 and a3. The guided light 202G exits fiber end 54 and enters microsphere 102 as (non-guided) light 202, which is strongly refracted by the strong surface curvature of the small microsphere. The result is focused light 202 that is directed to respective focuses F that reside along respective focus axes a1, a2 and a3. The focuses F are relatively close to the microsphere. The focused light 202 passes through the focus position F and then diverges, thereby forming divergent light 214. In the case where there is more than a single layer of microspheres 102, divergent light 214 from the first microsphere encounters then passes through one or more other microspheres 102, thereby increasing the amount of divergence of light 202.

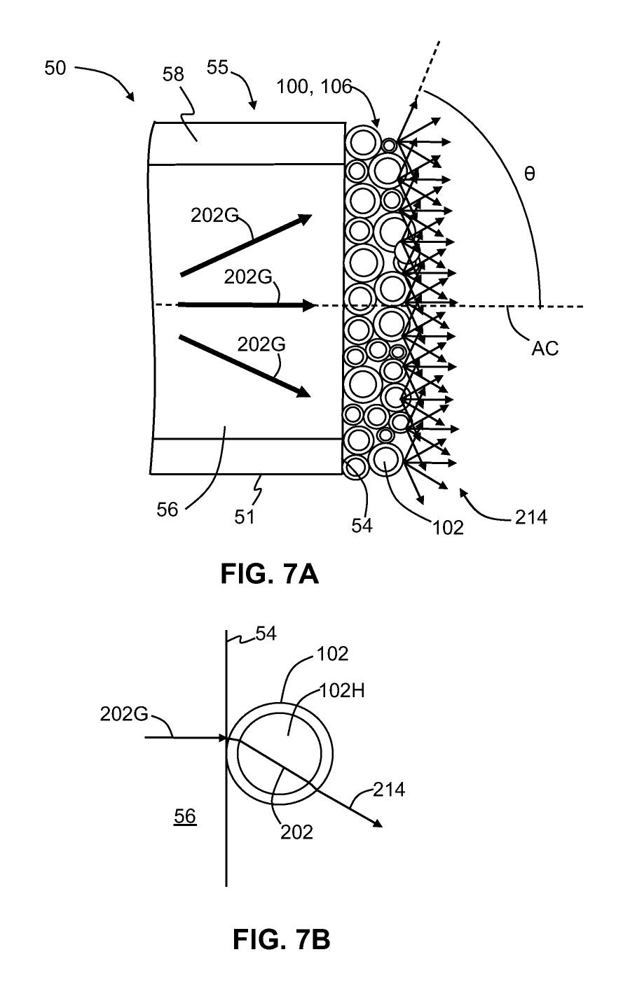

FIGS. 7A and 7B are similar to FIGS. 6A and 6B and illustrate the formation of divergent light 214 for the case where microspheres 102 are hollow, i.e., having a hollow center 102H, which in an example is assumed to be filled with air. FIG. 7B shows a single guided light ray 202G and how it exits fiber end 54 as non-guided light 202 that then refracts multiple times at the various interfaces of the hollow microsphere. The additional refractions (as compared to a solid microsphere) provide additional divergence of light 202.

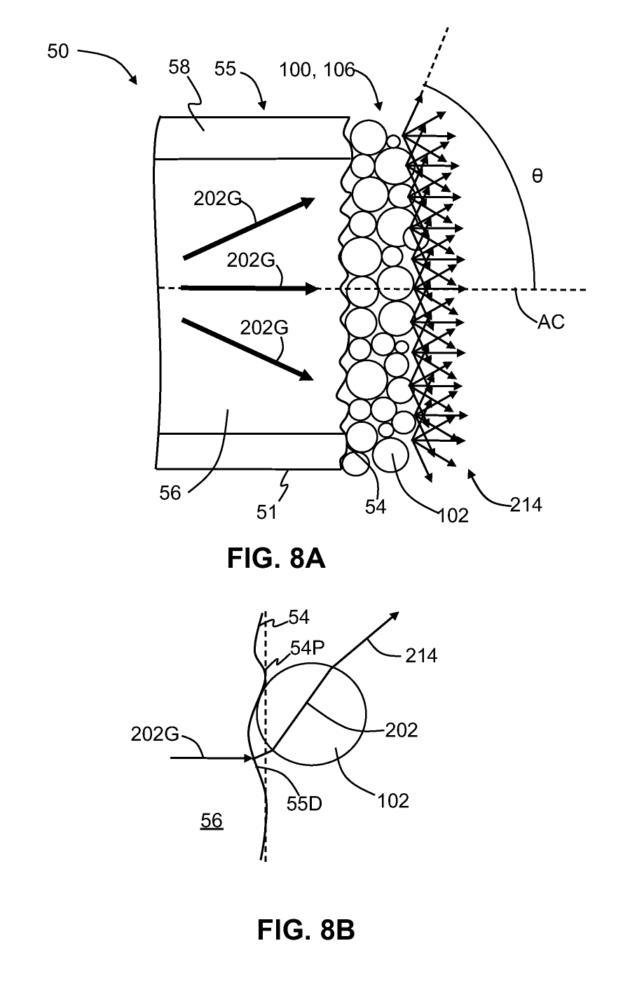

FIGS. 8A and 8B are similar to FIGS. 6A and 6B and illustrate and example of where fiber end 54 includes depressions (cavities) 54D and protrusions (ridges) 54P that serve to refract light 202 exiting the fiber end. The otherwise flat fiber end 54 is shown as a dashed line for reference. In an example, depressions 54D and protrusions 54P have a size that is substantially the same as that of microspheres 102 (i.e., substantially the same scale, or substantially the same radii of curvature) so that they are substantially non-scattering and redirect light substantially by refraction. The depressions 54D and protrusions 54P act as microlenses that work in combination with microspheres 102 to redirect light 202 to form redirected light 214. In another example, at least some of the depressions and protrusions 54D and 54P can have a scale on the order the wavelength of light 202 so that they scatter or diffuse the light exiting fiber end 54.

The depressions 54D and protrusions 54P can be formed on fiber end 54 using a number of techniques known in the art, including chemical etching or mechanical roughening. In an example, the chemical etching can employ a mask that allows for an etchant (e.g., an acid) to selectively act on fiber end 54. Example mask materials include an acid-resistant polymer or wax (e.g., wax microbeads).

The use of microspheres 102 allows for HDA fiber apparatus 50 to carry and emit a relatively large amount of optical power (e.g., up to about 300 milliwatts) because each microsphere only receives a small fraction of light 202 emitted from fiber end 202. In addition, the absence of a binding material in light-redirecting structure 106 avoids the problem of the high optical power burning off the binding material during use, especially during a biomedical-related use. A design that requires a binding material to bind the microspheres risks having the microspheres coming loose and being left behind in a biological medium if the optical power breaks down the binding material.

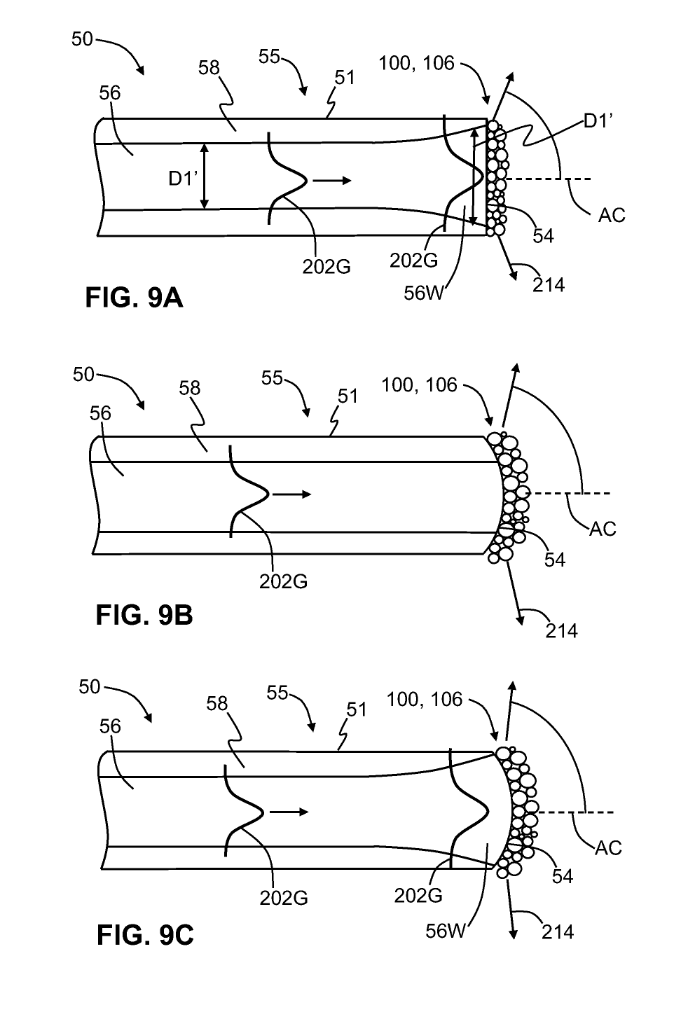

FIGS. 9A through 9C are cross-sectional views of bare end section 55 of HDA fiber apparatus 50 that illustrate several other example configurations of the HDA fiber apparatus. FIG. 9A shows an example where core 56 of optical fiber 51 widens adjacent fiber end 54 to form a widened core section 56W with a diameter D1′>D1, where D1′ is measured at the fiber end. In an example, widened core section 56W is defined by an adiabatic flaring of core 56, i.e., the core diameter D1 increases gradually to diameter D1′ so that the lateral extent of guided light 202G increases gradually with substantially no loss (e.g., without the coupling of light into cladding modes, etc.). In an example, widened core section 56W can be formed during the heating process used to fuse microspheres 102 to fiber end 54. In another example, bare end section 55 can be heat treated prior to applying microspheres 102 to fiber end 54. The heat treatment can be performed using laser system 140, arc fusion system 120 or other known heating systems and methods used in optical fiber processing.

In an example, the diameter D1′ of widened core section 56W is at least 5% greater than the core diameter D1, and in another example is at least 10% greater than the core diameter D1. The increased core diameter D1′ of widened core section 56W results in an increase in the mode-field diameter of guided light 202G at fiber end 54. The increased mode-field diameter serves to increase the divergence angle θ of divergent light 214 formed by light-redirecting structure 106.

FIG. 9B illustrates an example embodiment of HDA fiber apparatus 50 wherein fiber end 54 is curved rather than planar. In an example, the curvature is convex, as shown. In another example, the curvature can be concave. In an example, curved fiber end 54 can be formed using known techniques prior to the addition of microspheres 102 and the formation of light-redirecting structure 106.

FIG. 9C is similar to FIGS. 9A and 9B and shows an example of HDA fiber apparatus 50 that includes both the widened core section 56W of FIG. 9A and the curved fiber end 54 of FIG. 9B.

Light Source System

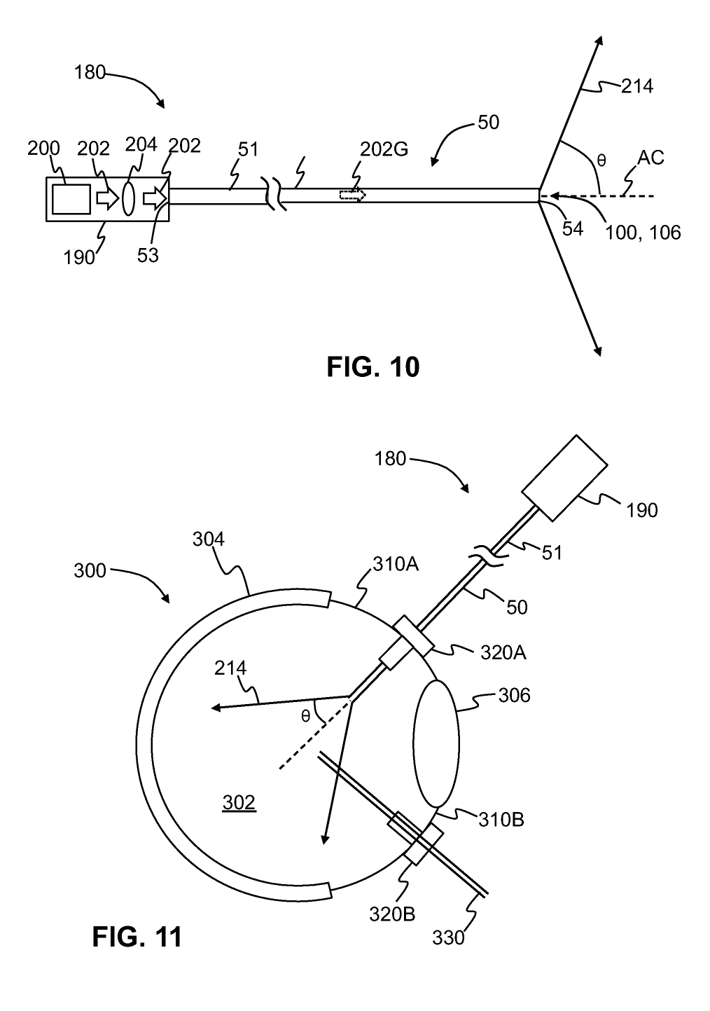

FIG. 10 is a schematic diagram of an example light source system 180 that includes HDA fiber apparatus 50. The light source system 180 includes a light source 190 optically coupled to optical fiber 51 at a proximal input end 53. The light source 190 includes a light emitter 200 that emits light 202. In an example, light emitter 200 includes a laser or a light-emitting diode. In an example, light emitter 200 emits non-polarized, broadband light 202 over at least a substantial portion of the visible spectrum (e.g., from 440 nm to 650 nm).

The light source 190 can also include an optical system 204 that couples light 202 from light emitter 200 into optical fiber 51 at input end 53, to form guided light 202G that travels down the optical fiber to fiber end (distal end) 54. As described above, guided light 202G exits fiber end 54 as light 202, which undergoes refraction by microspheres 102 in light-redirecting structure 106 to form divergent light 214.

FIG. 11 is a schematic diagram of light source system 180 of FIG. 10 operably arranged relative to an eye 300 for performing a biomedical procedure. The eye 300 includes a vitreous humor 302, a retina 304 and a lens 306. The vitreous humor 302 has first and second surface portions 310A and 310B on either side of lens 306. The output-end portion of HDA fiber apparatus 50 of light source system 180 is guided into vitreous humor 302 via a first cannula 320A operably arranged at surface portion 310A. A vitreous cutter probe 330 is inserted into vitreous humor 302 via a second cannula 3206 operably arranged at surface portion 310B. A saline infusion/suction device (not shown) is also inserted into vitreous humor 302 using a third cannula (not shown) operably at surface portion 310A or 310B.

The divergent light 214 from HDA fiber apparatus 50 serves to illuminate a wide section (volume) of vitreous humor 302 as well a wide surface portion of retina 304 so that the vitreous cutter probe 330 can be directed by a user to perform, for example, a vitrectomy or retinectomy.

The vitreous humor 302 is a fluid with a refractive index of 1.337, which is approximately the same as that of water. Consequently, the divergence angle θ of divergent light 214 from HDA fiber apparatus 50 is reduced when immersed in vitreous humor 302 (or any other liquid) as compared to an air environment. This reduction in the divergence angle θ when the output end of HDA fiber apparatus 50 is immersed in fluid is one reason why the divergence angle in air needs to be made very large. The vitreous humor 302 is one example of a biological fluid in which the light-redirecting structure 106 may be immersed during a biomedical procedure.

With reference again to FIG. 6A, the fused microspheres 102 of light-redirecting member 106 define interstices 103. When fiber end 54 is immersed in a liquid, such as the vitreous humor 302 of eye 300, the air trapped in these interstices can prevent the liquid from filling the interstices and further reducing the divergence angle θ. Note also that the absence of a binding material within interstices 103 also results in an improved divergence angle θ as compared to if such material were present.

As also noted above, the absence of an organic binding material in light-redirecting structure 106 avoids possible complications (including contaminations) relating to introducing such material into eye 300 or other part of the body.

It will be apparent to those skilled in the art that various modifications to the preferred embodiments of the disclosure as described herein can be made without departing from the spirit or scope of the disclosure as defined in the appended claims. Thus, the disclosure covers the modifications and variations provided they come within the scope of the appended claims and the equivalents thereto.

What is claimed is:

1. An high-divergence-angle (HDA) optical fiber apparatus, consisting of:
a multimode optical fiber having a glass core, a lower-index glass cladding surrounding the glass core, a proximal end and a distal end, wherein the optical fiber has a divergence angle θ′;
a light-redirecting structure operably disposed at the distal end and consisting of an array of fused glass microspheres having diameters in the range from 3 microns to 25 microns wherein the array is fused to the distal end and has between 1 layer and 10 layers of microspheres; and
wherein the light-redirecting structure defines a divergence angle θ, wherein θ≥2θ′.

2. The HDA optical fiber apparatus according to claim 1, wherein the optical fiber has a core diameter D1 of nominally 40 microns and a cladding outer diameter D2 of nominally 50 microns.

3. The HDA optical fiber apparatus according to claim 1, wherein the glass microspheres, the glass core and the glass cladding are each made of a silica-based glass.

4. The HDA optical fiber apparatus according to claim 1, wherein the array has between 1 layer and 6 layers of the microspheres.

5. The HDA optical fiber apparatus according to claim 1, wherein the microspheres are solid.

6. The HDA optical fiber apparatus according to claim 1, wherein θ≥3θ′.

7. The HDA optical fiber apparatus according to claim 1, wherein the core has a diameter D1 and includes adjacent the distal end a widened core section that has a diameter D1′ that is at least 5% greater than the core diameter D1.

8. The HDA optical fiber apparatus according to claim 1, wherein the distal end of the optical fiber is curved.

9. The HDA optical fiber apparatus according to claim 1, wherein the distal end of the optical fiber includes depressions and protrusions that have a size substantially the same as the glass microspheres.

10. The HAD optical fiber apparatus according to claim 1, wherein the fused microspheres define air-filled interstices within the light-redirecting structure.

11. A light source system, comprising:
the HDA optical fiber apparatus according to claim 1; and
a light source optically coupled to the proximal end of the optical fiber and that includes a light emitter that emits light that is coupled into the optical fiber, wherein the light is emitted from the light-redirecting structure as divergent light over the second divergence angle θ.

12. The light source system according to claim 11, wherein the light emitter emits non-polarized visible light in the range from 440 nm to 650 nm.

13. A method of forming a high-divergence-angle (HDA) optical fiber apparatus using a multimode optical fiber having a glass core, a lower-index glass cladding surrounding the glass core, a proximal end and a distal end, wherein the optical fiber has a first divergence angle θ′, the method comprising:
arranging an array of glass microspheres adjacent the distal end,
the microspheres having diameters in the range from 3 microns to 25 microns; and
applying heat to the microspheres and to the distal end to fuse the microspheres to each other and to the distal end to form a light-redirecting structure that consists only of the microspheres and that defines a second divergence angle θ, wherein θ≥2θ′.

14. The method according to claim 13, further comprising:
prior to the act of applying heat, using a binding material to bind the microspheres to each other and to the distal end of the optical fiber; and
wherein the act of applying heat includes burning off substantially all of the binding material.

15. The method according to claim 13, wherein the optical fiber has a core diameter of nominally 40 microns and a cladding outer diameter of nominally 50 microns.

16. The method according to claim 13, wherein the glass microspheres, the core and the cladding are made of a silica-based glass.

17. The method according to claim 13, wherein the array has between 1 layer and 6 layers of microspheres.

18. The method according to claim 13, wherein the microspheres are solid.

19. The method according to claim 13, wherein the fused microspheres define air-filled interstices within the light-redirecting structure.

20. The method according to claim 13, wherein the core has a diameter D1 and further including a widened core section adjacent the distal end, the widened core section having a diameter D1′ that is at least 5% greater than the core diameter D1.

21. The method according to claim 13, further comprising forming the distal end of the optical fiber to have a convex curvature.

22. The method according to claim 13, further comprising forming protrusions and depressions on the distal end of the optical fiber, wherein the protrusion and depressions have a size substantially the same as the microspheres.

23. The method according to claim 13, further comprising:
operably arranging a light source relative to the proximal end of the optical fiber and emitting a divergent light beam from the light-redirecting structure.

24. The method according to claim 23, wherein the light source emits non-polarized broadband visible light over a spectral range from 440 nm to 650 nm.

25. The method according to claim 23, further including immersing the light-redirecting structure in a biological fluid.

26. A light source system that emits divergent light of a visible wavelength λ, comprising:
a multimode optical fiber having a proximal end and a distal end, and a divergence angle θ′;
a light-redirecting structure operably disposed at the distal end and consisting of an array of fused glass microspheres having diameters in the range from 3 microns to 25 microns, wherein the light-redirecting structure is fused to the distal end and has between 1 layer and 10 layers of microspheres and defines a divergence angle θ, wherein θ≥2θ′; and
a light source optically coupled to the proximate end of the optical fiber and that emits light of the visible wavelength λ.
